(19) United States
(12) Patent Application Publication
(10) Pub. No.: US 2019/0030025 A1
(43) Pub. Date: Jan. 31, 2019

(54) FOSTEMSAVIR FOR USE IN HEAVILY TREATMENT-EXPERIENCED HIV-1 INFECTED INDIVIDUALS

(71) Applicant: ViiV HEALTHCARE UK (NO.5) LIMITED, Brentford, Middesex (GB)

(72) Inventors: Dennis Michael GRASELA, Wallingford, CT (US); George HANNA, Wallingford, CT (US); Mark R. KRYSTAL, Wallingford, CT (US); Max LATAILLADE, Wallingford, CT (US)

(21) Appl. No.: 16/071,981

(22) PCT Filed: Feb. 2, 2017
(86) PCT No.: PCT/IB2017/050571

§ 371 Date: Jul. 23, 2018

Related U.S. Application Data
(60) Provisional application No. 62/291,302, filed on Feb. 4, 2016.

(51) Int. Cl. A61K 31/496; A61K 31/635; A61K 31/5365; A61K 31/662; A61K 31/505; A61K 31/439; A61K 45/06

(52) U.S. Cl. CPC A61K 31/496; A61K 31/635; A61K 31/5365; A61K 45/06; A61K 31/505; A61K 31/439; A61K 31/662

(57) ABSTRACT

A method of attaining virologic suppression in a heavily treatment-experienced (HTE) individual infected with the HIV-1 virus involves administering to the individual a treatment regimen comprising the drug fostemsavir together with an optimized background therapy (OBT). This treatment regimen should maintain virologic suppression to a plasma HIV-1 RNA level of less than 200 copies (c)/mL for an extended period.

### FIELD OF THE INVENTION

The present invention relates to a method of treatment of HIV-1 infection, and more particularly, to a method of treating heavily treatment-experienced patients who may have also failed to achieve or maintain virologic suppression. The invention also relates to the treatment regimen herein set forth.

### BACKGROUND OF THE INVENTION

With the introduction of combination antiretroviral therapy to treat HIV-1 infection, mortality from the acquired immune deficiency syndrome (AIDS) has declined markedly. With this reduction in mortality, the number of people living with HIV-1 infection worldwide has increased, and HIV-1 infection is now considered a chronic disease requiring life-long therapy. Despite the availability of different classes of antiretroviral (ARV) agents providing a variety of treatment options, treatment failure continues to occur as a result of ARV drug resistance, drug-associated toxicity and tolerability problems, and poor adherence. Treatment failure may result in selection of a virus with resistance to one or more antiretroviral agent(s). Furthermore, resistance mutations selected by one antiretroviral often confer resistance to multiple drugs in the same class, significantly limiting future therapeutic options. Later-line regimens often lack the convenience and tolerability of first-line drugs, which in turn can further exacerbate non-adherence and non-compliance.

In particular, heavily-treatment-experienced (HTE) patients who by definition have failed multiple ARV classes/regimens, have very few remaining therapeutic options (≤2 remaining fully active ARVs available to be combined in a suppressive regimen, based on documented resistance testing), and are not uncommonly on ARV combinations that are highly individualized and may lack efficacy, safety and tolerability profiles of agents traditionally used in earlier lines of therapy. These individuals may be at dangerous risk of, or may have already achieved virologic failure. The NIH defines virologic failure as the inability to achieve or maintain suppression of viral replication to an HIV-1 RNA level of <200 copies (c)/mL. Without reversal, virologic failure will lead to full blown AIDS, the rise of opportunistic infections, and ultimately death.

Bristol-Myers Squibb has now developed fostemsavir, a novel attachment inhibitor prodrug, as a mono-entity for use in treating HIV-infected individuals. Fostemsavir has been found to be generally safe and well-tolerated, possesses good efficacy, and offers a unique mechanism of action. By blocking the gp120 receptor of the virus, it prevents initial viral attachment to the host CD4+ T cell and entry into the host immune cell; its method of action is a first for HIV drugs. Fostemsavir has the structure shown below:

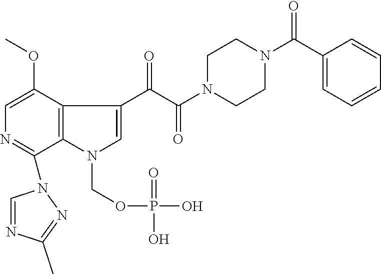 [SMILES: COc1cnc(-n2cnc(C)n2)c2c1c(C(=O)C(=O)N1CCN(C(=O)c3ccccc3)CC1)cn2COP(=O)(O)O]

and is set forth and claimed in U.S. Pat. No. 7,745,625, incorporated herein by reference. Fostemsavir is a phosphate prodrug and its parent compound has the structure below:

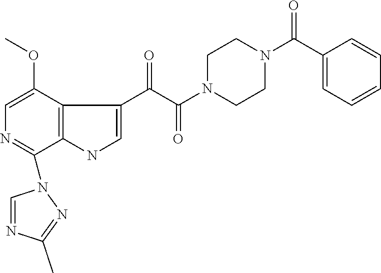 [SMILES: COc1cnc(-n2cnc(C)n2)c2[nH]cc(C(=O)C(=O)N3CCN(C(=O)c4ccccc4)CC3)c12]

and is set forth and claimed in U.S. Pat. No. 7,354,924, also incorporated herein by reference.

There is an ongoing need for new classes of antiretroviral drugs capable of providing potent, durable antiviral activity not only for treatment-naïve individuals, i.e. those who have never been on a regimen or “cocktail” of HIV medications, but also for treatment of heavily treatment-experienced individuals who may be victims of antiretroviral-resistant viruses, which are very often a far more difficult subset of patients to treat. New therapies should aim to be convenient and have favorable tolerability and safety profiles in order to provide greater treatment options, in particular for heavily treatment-experienced subjects. New therapies should also allow patients to attain and maintain virologic suppression for extended periods of time. Because fostemsavir targets a different step of the HIV-1 viral lifecycle, it offers promise for individuals infected with HIV that have become highly resistant to other HIV drugs. Since gp120 is a highly conserved area of the virus, the drug is unlikely to promote resistance to itself via generation of CD4-independent virus. Thus, there is promise in using fostemsavir for heavily treatment-experienced individuals. However, because of the evolving nature of the HIV virus and its impact on those it has infected, treatment regimens have proven to be highly unpredictable to date. This area is often fraught with more disappointment than success.

### SUMMARY OF THE INVENTION

In a first embodiment, the invention is directed to a method of attaining virologic suppression in a heavily treatment-experienced (HTE) individual infected with the HIV-1 virus, in which a treatment regimen comprising the drug fostemsavir, together with an optimized background therapy (OBT) is administered to said individual.

The present invention is directed to these, as well as other important ends, hereinafter described.

### DETAILED DESCRIPTION OF THE EMBODIMENTS

HIV/AIDS related morbidity and mortality continues to be a significant epidemic internationally. A substantial number of HIV-infected individuals have failed prior therapies (for reasons including but not limited to safety, resistance, and tolerability). The therapeutic goal for treatment-experienced patients who are failing current therapy is to construct a new regimen that contains at least two (and preferably three) fully active ARVs that re-establish virologic suppression. An ARV with a novel mechanism of action may meet the criteria as a fully active agent. Finally, treatment-experience individuals may have a longer history of exposure to various ARVs with short and longer term safety problems. There is a need for new agents with novel mechanisms of action (MOAs) and favorable efficacy, safety, and tolerability profiles in heavily treatment-experienced adults. Fostemsavir, an attachment inhibitor prodrug with a novel mechanism of action has shown favorable efficacy, safety and tolerability in HIV-1 infected subjects who are treatment-naïve, in combination with an optimal ARV backbone. The purpose of this invention is thus to fulfill the unmet medical need of treating HIV-1 infected patients who are highly-treatment-experienced, and who may be at risk of not attaining virologic suppression.

Thus, the invention provides a method of attaining virologic suppression in a heavily-treatment-experienced individual which comprises administering to that individual a treatment regimen comprising the drug fostemsavir together with an optimized background therapy (OBT). The infected individual, prior to administration of the treatment regimen, will preferably have a plasma HIV-1 RNA level of greater than about 400 copies (c)/mL. Other individuals, prior to administration of the treatment regimen may have a plasma HIV-1 RNA level greater than about 1000 c/mL. In a further embodiment, the HTE individuals, prior to initiation of said treatment regimen, will have a viral load greater than about 5000 c/mL, and in some instances, equal to or greater than about 10,000 c/mL. Thus, it is preferred that the infected individual will be a heavily-treatment-experienced individual who has not been able to achieve or maintain virologic suppression.

The drug fostemsavir is administered herein to the heavily-treatment-experienced individual. Preferably, a dose of about 1200 mg of fostemsavir is administered to the individual daily. This dose can be in the form of one 1200 mg dose, or more preferably, two 600 mg. doses daily. Other dosing regimens are within the purview of the skilled artisan.

As stated herein, the drug fostemsavir is administered with an optimized background therapy, or OBT. The OBT is comprised of at least one, and more preferably, at least two other HIV drug medications. These other HIV drug medications are preferably active antiretrovirals (ARVs) which the infected individual has not already failed. Those skilled in the art will be able to recognize which are failed medications by documented resistance testing, as well as other testing methods available in the art.

An exemplary, non-limiting listing of HIV medications is provided herein, which may be dosed according to established protocol is set forth herein:

ANTIVIRALS

| Drug Name | Manufacturer | Indication |
|---|---|---|
| 097 | Hoechst/Bayer | HIV infection, |
| | | AIDS, ARC |
| | | (non-nucleoside |
| | | reverse transcriptase |
| | | (RT) |
| | | inhibitor) |
| Amprenavir | Glaxo Wellcome | HIV infection, |
| 141 W94 | | AIDS, ARC |
| GW 141 | | (protease inhibitor) |
| Abacavir (1592U89) | Glaxo Wellcome | HIV infection, |
| GW 1592 | | AIDS, ARC |
| | | (RT inhibitor) |
| Acemannan | Carrington Labs | ARC |
| | (Irving, TX) | |
| Acyclovir | Burroughs Wellcome | HIV infection, AIDS, |
| | | ARC |
| AD-439 | Tanox Biosystems | HIV infection, AIDS, |
| | | ARC |
| AD-519 | Tanox Biosystems | HIV infection, AIDS, |
| | | ARC |
| Adefovir dipivoxil | Gilead Sciences | HIV infection |
| AL-721 | Ethigen | ARC, PGL |
| | (Los Angeles, CA) | HIV positive, AIDS |
| Alpha Interferon | Glaxo Wellcome | Kaposi's sarcoma, |
| | | HIV in combination w/Retrovir |
| Ansamycin | Adria Laboratories | ARC |
| LM 427 | (Dublin, OH) | |
| | Erbamont | |
| | (Stamford, CT) | |
| Antibody which | Advanced Biotherapy | AIDS, ARC |
| Neutralizes pH | Concepts | |
| Labile alpha aberrant | (Rockville, MD) | |
| Interferon | | |
| AR177 | Aronex Pharm | HIV infection, AIDS, |
| | | ARC |
| Beta-fluoro-ddA | Nat'l Cancer Institute | AIDS-associated |
| | | diseases |
| BMS-234475 | Bristol-Myers Squibb/ | HIV infection, |
| (CGP-61755) | Novartis | AIDS, ARC |
| | | (protease inhibitor) |
| CI-1012 | Warner-Lambert | HIV-1 infection |
| Cidofovir | Gilead Science | CMV retinitis, |
| | | herpes, papillomavirus |
| Curdlan sulfate | AJI Pharma USA | HIV infection |
| Cytomegalovirus | MedImmune | CMV retinitis |
| Immune globin | | |
| Cytovene | Syntex | Sight threatening |
| Ganciclovir | | CMV |
| | | peripheral CMV |
| | | retinitis |
| Darunavir | Tibotec-J & J | HIV infection, AIDS, ARC |
| | | (protease inhibitor) |
| Delaviridine | Pharmacia-Upjohn | HIV infection, |
| | | AIDS, ARC |
| | | (RT inhibitor) |
| Dextran Sulfate | Ueno Fine Chem. | AIDS, ARC, HIV |
| | Ind. Ltd. (Osaka, | positive |
| | Japan) | asymptomatic |
| ddC | Hoffman-La Roche | HIV infection, AIDS, |
| Dideoxycytidine | | ARC |
| ddI | Bristol-Myers Squibb | HIV infection, AIDS, |
| Dideoxyinosine | | ARC; combination |
| | | with AZT/d4T |
| DMP-450 | AVID | HIV infection, |
| | (Camden, NJ) | AIDS, ARC |
| | | (protease inhibitor) |
| Efavirenz | Bristol Myers Squibb | HIV infection, |
| (DMP 266, SUSTIVA ®) | | AIDS, ARC |
| (−)6-Chloro-4-(S)- | | (non-nucleoside RT |
| cyclopropylethynyl- | | inhibitor) |
| 4(S)-trifluoro- | | |
| methyl-1,4-dihydro- | | |
| 2H-3,1-benzoxazin- | | |
| 2-one, STOCRINE | | |
| EL10 | Elan Corp, PLC | HIV infection |
| | (Gainesville, GA) | |
| Etravirine | Tibotec/J & J | HIV infection, AIDS, ARC |
| | | (non-nucleoside |
| | | reverse transcriptase |
| | | inhibitor) |
| Famciclovir | Smith Kline | herpes zoster, |
| | | herpes simplex |
| GS 840 | Gilead | HIV infection, |
| | | AIDS, ARC |
| | | (reverse transcriptase |
| | | inhibitor) |
| HBY097 | Hoechst Marion | HIV infection, |
| | Roussel | AIDS, ARC |
| | | (non-nucleoside |
| | | reverse transcriptase |
| | | inhibitor) |
| Hypericin | VIMRx Pharm. | HIV infection, AIDS, |
| | | ARC |
| Recombinant Human | Triton Biosciences | AIDS, Kaposi's |
| Interferon Beta | (Almeda, CA) | sarcoma, ARC |
| Interferon alfa-n3 | Interferon Sciences | ARC, AIDS |
| Indinavir | Merck | HIV infection, AIDS, |
| | | ARC, asymptomatic |
| | | HIV positive, also in |
| | | combination with |
| | | AZT/ddI/ddC |
| ISIS 2922 | ISIS Pharmaceuticals | CMV retinitis |
| KNI-272 | Nat'l Cancer Institute | HIV-assoc. diseases |
| Lamivudine, 3TC | Glaxo Wellcome | HIV infection, |
| | | AIDS, ARC |
| | | (reverse |
| | | transcriptase |
| | | inhibitor); also |
| | | with AZT |
| Lobucavir | Bristol-Myers Squibb | CMV infection |
| Nelfinavir | Agouron | HIV infection, |
| | Pharmaceuticals | AIDS, ARC |
| | | (protease inhibitor) |
| Nevirapine | Boeheringer | HIV infection, |
| | Ingleheim | AIDS, ARC |
| | | (RT inhibitor) |
| Novapren | Novaferon Labs, Inc. | HIV inhibitor |
| | (Akron, OH) | |
| Peptide T | Peninsula Labs | AIDS |
| Octapeptide | (Belmont, CA) | |
| Sequence | | |
| Trisodium | Astra Pharm. | CMV retinitis, HIV |
| Phosphonoformate | Products, Inc. | infection, other CMV |
| | | infections |
| PNU-140690 | Pharmacia Upjohn | HIV infection, |
| | | AIDS, ARC |
| | | (protease inhibitor) |
| Probucol | Vyrex | HIV infection, AIDS |
| RBC-CD4 | Sheffield Med. | HIV infection, |
| | Tech (Houston, TX) | AIDS, ARC |
| Ritonavir | Abbott | HIV infection, |
| | | AIDS, ARC |
| | | (protease inhibitor) |
| Saquinavir | Hoffmann- | HIV infection, |
| | LaRoche | AIDS, ARC |
| | | (protease inhibitor) |
| Stavudine; d4T | Bristol-Myers Squibb | HIV infection, AIDS, |
| Didehydrodeoxy- | | ARC |
| Thymidine | | |
| Tipranavir | Boehringer Ingelheim | HIV infection, AIDS, ARC |
| | | (protease inhibitor) |
| Valaciclovir | Glaxo Wellcome | Genital HSV & CMV |
| | | infections |
| Virazole | Viratek/ICN | asymptomatic HIV |
| Ribavirin | (Costa Mesa, CA) | positive, LAS, ARC |
| VX-478 | Vertex | HIV infection, AIDS, |
| | | ARC |
| Zalcitabine | Hoffmann-LaRoche | HIV infection, AIDS, |
| | | ARC, with AZT |
| Zidovudine; AZT | Glaxo Wellcome | HIV infection, AIDS, ARC, |
| | | Kaposi's sarcoma, in |
| | | combination with other |
| | | therapies |
| Tenofovir disoproxil, | Gilead | HIV infection, |
| fumarate salt (VIREAD ®) | | AIDS, |
| | | (reverse transcriptase |
| | | inhibitor) |
| EMTRIVA ® | Gilead | HIV infection, |
| (Emtricitabine) (FTC) | | AIDS, |
| | | (reverse transcriptase |
| | | inhibitor) |
| COMBIVIR ® | GSK | HIV infection, |
| | | AIDS, |
| | | (reverse transcriptase |
| | | inhibitor) |
| Abacavir succinate | GSK | HIV infection, |
| (or ZIAGEN ®) | | AIDS, |
| | | (reverse transcriptase |
| | | inhibitor) |
| REYATAZ ® | Bristol-Myers Squibb | HIV infection |
| (or atazanavir) | | AIDs, protease |
| | | Protease inhibitor |
| FUZEON ® | Roche/Trimeris | HIV infection |
| (Enfuvirtide or T-20) | | AIDs, viral Fusion |
| | | inhibitor |
| LEXIVA ® | GSK/Vertex | HIV infection |
| (or Fosamprenavir calcium) | | AIDs, viral protease |
| | | inhibitor |
| Selzentry | Pfizer | HIV infection |
| Maraviroc; (UK 427857) | | AIDs, (CCR5 antagonist, in |
| | | development) |
| Trizivir ® | GSK | HIV infection |
| | | AIDs, (three drug combination) |
| Sch-417690 (vicriviroc) | Schering-Plough | HIV infection |
| | | AIDs, (CCR5 antagonist, in |
| | | development) |
| TAK-652 | Takeda | HIV infection |
| | | AIDs, (CCR5 antagonist, in |
| | | development) |
| GSK 873140 | GSK/ONO | HIV infection |
| (ONO-4128) | | AIDs, (CCR5 antagonist, |
| | | in development) |
| Integrase Inhibitor | Merck | HIV infection |
| MK-0518 | | AIDs |
| Raltegravir | | |
| TRUVADA ® | Gilead | Combination of Tenofovir |
| | | (disoproxil fumarate salt) |
| | | VIREAD ® and EMTRIVA ® |
| | | (Emtricitabine) |
| Integrase Inhibitor | Gilead/Japan Tobacco | HIV Infection |
| GS917/JTK-303 | | AIDs |
| Elvitegravir | | |
| Integrase Inhibitor | | |
| Triple drug combination | Gilead/Bristol-Myers Squibb | Combination of Tenofovir |
| ATRIPLA ® | | disoproxil fumarate salt |
| | | (VIREAD ®), EMTRIVA ® |
| | | (Emtricitabine), and |
| | | SUSTIVA ® (Efavirenz) |
| FESTINAVIR ® | Oncolys BioPharma | HIV infection |
| 4′-ethynyl-d4T | BMS | AIDs |
| in development | | |
| CMX-157 | Chimerix | HIV infection |
| Lipid conjugate of | | AIDs |
| nucleotide tenofovir | | |
| GSK1349572 | GSK | HIV infection |
| Integrase inhibitor | | AIDs |
| dolutegravir | | |
| S/GSK1265744 | GSK | HIV infection |
| Integrase inhibitor | | AIDs |

Of the foregoing, darunavir, dolutegravir, tenofovir, etravirine, and maraviroc may be preferred in some embodiments.

As set forth above, the NIH has defined virologic failure in the context of HIV-1 infection as the inability to achieve or maintain suppression of viral replication to an HIV-1 RNA level of <200 copies (c)/mL. Thus, an embodiment of the invention herein set forth is to attain virologic suppression in a heavily-treatment-experienced individual such that the HIV-1 RNA level is less than about 200 c/mL. More preferably, the HIV-1 RNA level will be less than about 100 c/mL. Even more preferably, the HIV-1 RNA level will be less than about 40 c/mL. An HIV-1 RNA level of less than about 20 c/mL is also within the scope of the invention.

It is also important for an infected patient undergoing HIV treatment to maintain virologic suppression for an extended period on the same regimen of fostemsavir plus the OBT of 1-2 additional HIV medications. Therefore, it is also an embodiment of the invention that virologic suppression is maintained for at least about 24 weeks of the treatment regimen. It is further preferred that the virologic suppression be maintained for at least about 48 weeks of the treatment regimen herein set forth. In addition, it is preferred that virologic suppression be maintained for at least about 96 weeks duration of the treatment regimen herein. Even more preferably, virologic suppression should be maintained for at least about 2 years, and more preferably for at least about 5 years of the treatment regimen.

The foregoing description is merely illustrative and should not be understood to limit the scope or underlying principles of the invention in any way. Indeed, various modifications of the invention, in addition to those shown and described herein, will become apparent to those skilled in the art from the following examples and the foregoing description. Such modifications are also intended to fall within the scope of the appended claims.

What is claimed is:

1. A method of attaining virologic suppression in a heavily treatment-experienced (HTE) individual infected with the HIV-1 virus, which comprises administering to said individual a treatment regimen comprising the drug fostemsavir together with an optimized background therapy (OBT).

2. The method of claim 1, wherein said individual prior to said administration has a plasma HIV-1 RNA level of greater than about 400 copies (c)/mL.

3. The method of claim 1, wherein said OBT comprises 1-2 other HIV drug medications.

4. The method of claim 3, wherein said OBT comprises at least 1 other HIV drug medication.

5. The method of claim 3, wherein said OBT comprises at least 2 other HIV drug medications.

6. The method of claim 3, wherein said other HIV drug medications are active antiretrovirals (ARVs) which said individual has not previously failed.

7. The method of claim 1, wherein a total of 1200 mg of said fostemsavir is administered daily.

8. The method of claim 7, wherein said fostemsavir is administered in two 600 mg doses (one 600mg tablet twice daily).

9. The method of claim 1, wherein said virologic suppression is a plasma HIV-1 RNA level of less than 40 c/mL.

10. The method of claim 2, wherein said virologic suppression is a plasma HIV-1 RNA level of less than 40 c/mL.

11. The method of claim 9, wherein said virologic suppression is recorded at least at week 24 of said treatment regimen.

12. The method of claim 10, wherein said virologic suppression is recorded at least at week 24 of said treatment regimen.

13. The method of claim 11, wherein said virologic suppression is recorded at least at week 48 of said treatment regimen.

14. The method of claim 12, wherein said virologic suppression is recorded at least at week 48 of said treatment regimen.

15. The method of claim 13, wherein said virologic suppression is recorded at least at week 96 of said treatment regimen.

16. The method of claim 14, wherein said virologic suppression is recorded at least at week 96 of said treatment regimen.

17. The method of claim 6, wherein said other HIV drug medications are selected from the group of darunavir, dolutegravir, tenofovir, etravirine, and maraviroc.
